(19) United States
(12) Patent Application Publication
(10) Pub. No.: US 2006/0182812 A1
(43) Pub. Date: Aug. 17, 2006

(54) Antibacterial compositions and antibacterial products

(76) Inventor: Yasuharu Ono, Aichi (JP)

Correspondence Address: SUGHRUE MION, PLLC, 2100 PENNSYLVANIA AVENUE, N.W., SUITE 800, WASHINGTON, DC, 20037, US

(21) Appl. No.: 10/542,732

(22) PCT Filed: Jan. 19, 2004
(86) PCT No.: PCT/JP04/00362

§ 371 Date: Jul. 20, 2005

(30) Foreign Application Priority Data
Jan. 20, 2003 (JP) 2003-10588

(51) Int. Cl. A61K 33/42

(52) U.S. Cl. 424/604

(57) ABSTRACT

The present invention has an object of improving the processing suitability of an antimicrobial product such as fiber or a film to which is added an antimicrobial composition that includes a silver-based antimicrobial agent such as silver ion-containing tetravalent metal phosphate-based antimicrobial particles. The present invention is an antimicrobial composition that includes tetravalent metal phosphate-based antimicrobial particles represented by Formula (1) and inorganic compound particles having a Mohs hardness of equal to or less than 6, and the maximum particle size of the antimicrobial particles and the inorganic compound particles is substantially equal to or less than 10 μm.
AgₐQ_{b}M₂(PO₄)₃.nH₂O (1)

In the formula, Q denotes at least one type of ion selected from the group consisting of an alkali metal ion, an alkaline earth metal ion, ammonium ion, and hydrogen ion, M is a tetravalent metal ion, n is a number satisfying 0≦n≦6, a and b are both positive numbers, m is the valence of Q, and a+mb=1. Furthermore, the present invention is an antimicrobial product that contains the antimicrobial composition.

### TECHNICAL FIELD

The present invention relates to an antimicrobial composition and, more particularly, to an antimicrobial composition that can improve the processability of a fiber or a film to which the antimicrobial composition is added.

### BACKGROUND ART

Tetravalent metal phosphate-based antimicrobial agents have excellent antimicrobial properties, safety, and heat resistance, and they are therefore added to various type of resins and utilized in a wide range of applications as additives that impart long-lasting antimicrobial properties to commonplace resin moldings such as fibers, films, and various types of containers (ref., e.g. JP-A-3-083905, JP-A-7-304620, and JP-A-2002-309445 (JP-A denotes a Japanese unexamined patent application publication)).

However, when a molding is produced by adding the tetravalent metal phosphate-based antimicrobial agent to a resin, there is the problem that an area of equipment that is in running contact with the molding is easily worn. For example, in the case of a fiber, the fiber is in running contact with a guide, etc. in a spinning step or a posttreatment step, and since the contact point during this process is concentrated on one area of the guide, etc., even when a guide made of a high hardness ceramic such as alumina or zirconia is used, there is the problem that wear easily occurs.

As a countermeasure against the wear of a guide, etc. during spinning of fiber, a method has been proposed in which the fiber is made of a core-sheath composite filament, and inorganic particles are added to the core part (ref., e.g. JP-A-8-144151 and JP-A-62-57920). However, in this method, since the tetravalent metal phosphate-based antimicrobial agent is added to the core part of the fiber, it is difficult for an antimicrobial effect to be exhibited.

A method has also been proposed for suppressing wear by improving particle dispersibility by specifying the composition and the structure of an antimicrobial zeolite support (ref., e.g. JP-A-8-151515), but since the antimicrobial zeolite is easily degraded due to poor chemical resistance, washing resistance, light fastness, etc., the antimicrobial properties are not durable, and excellent performance such as that provided by the tetravalent metal phosphate-based antimicrobial agent cannot be obtained.

A process for producing a fiber in which inorganic particles are added to a fiber body has been disclosed (ref., e.g. JP-A-2001-159024), but there is no description or suggestion concerning an inorganic antimicrobial agent.

### DISCLOSURE OF INVENTION

It is an object of the present invention to improve the suitability for processing into an antimicrobial product of a fiber or film to which has been added a silver-based antimicrobial agent-containing antimicrobial composition such as silver ion-containing tetravalent metal phosphate-based antimicrobial particles.

As a result of an intensive investigation by the present inventor in order to attain the above-mentioned object, it has been found that the above-mentioned problems can be solved by an antimicrobial composition comprising particles of a specified tetravalent metal phosphate-based antimicrobial agent and inorganic compound particles having a Mohs hardness of equal to or less than 6, the maximum particle size of both types of particles being substantially equal to or less than 10 μm, and the present invention has thus been accomplished. That is, the above-mentioned specified tetravalent metal phosphate-based antimicrobial particles are represented by Formula (1) below.
AgₐQ_{b}M₂(PO₄)₃.nH₂O (1)

In the formula, Q is at least one type of ion selected from the group consisting of an alkali metal ion, an alkaline earth metal ion, ammonium ion, and hydrogen ion, M is a tetravalent metal ion, n is a number satisfying 0≦n≦6, a and b are both positive numbers, m is the valence of Q, and a+mb=1.

Furthermore, this has also led to the invention of an antimicrobial product comprising the above-mentioned antimicrobial composition.

The present invention has been accomplished based on the above-mentioned understandings, and representative examples thereof are cited below.

1. An antimicrobial composition comprising a tetravalent metal phosphate-based antimicrobial particles represented by Formula (1) above and inorganic compound particles having a Mohs hardness of equal to or less than 6, the maximum particle size of these particles being substantially equal to or less than 10 μm.

2. The antimicrobial composition according to 1 above, wherein the tetravalent metal phosphate-based antimicrobial particles and the inorganic compound particles have an average particle size of 0.1 to 5 μm.

3. The antimicrobial composition according to 1 above, wherein the average particle size of the inorganic compound particles is smaller than the average particle size of the tetravalent metal phosphate-based antimicrobial particles.

4. The antimicrobial composition according to 1 above, wherein the inorganic compound particles are anatase titanium dioxide having no photocatalytic activity.

5. The antimicrobial composition according to 1 above, wherein the mixing ratio of the tetravalent metal phosphate-based antimicrobial particles and the inorganic compound particles is 95:5 to 10:90.

6. An antimicrobial product comprising the antimicrobial composition according to any one of 1 to 5 above.

7. The antimicrobial product according to 6 above, wherein the antimicrobial product is an antimicrobial fiber or an antimicrobial film.

### BEST MODE FOR CARRYING OUT THE INVENTION

The present invention is explained in detail below.

Tetravalent Metal Phosphate-Based Antimicrobial Agent

The silver ion-containing tetravalent metal phosphate-based antimicrobial agent in the present invention (simply called a ‘tetravalent metal phosphate-based antimicrobial agent’ in the present invention) is one represented by Formula (1) above.

The tetravalent metal phosphate represented by Formula (1) is a crystalline compound belonging to the space group R3c, with the constituent ions forming a three-dimensional network structure.

Q in Formula (1) is at least one type of ion selected from the group consisting of an alkali metal ion, an alkaline earth metal ion, ammonium ion, and hydrogen ion; preferred specific examples include alkali metal ions such as lithium, sodium, and potassium, alkaline earth metal ions such as magnesium and calcium, and hydrogen ion, and among these ions lithium, sodium, ammonium ion, and hydrogen ion are preferable from the viewpoint of compound stability and availability at low cost.

M in Formula (1) is a tetravalent metal ion, and preferred specific examples include zirconium ion, titanium ion, and tin ion. Zirconium ion and titanium ion are more preferable when compound safety is taken into consideration, and a particularly preferable tetravalent metal ion is zirconium ion.

As specific examples of Formula (1), those below can be cited.
Ag_{0.005}Li_{0.995}Zr₂(PO₄)₃
Ag_{0.01}(NH₄)_{0.99}Zr₂(PO₄)₃
Ag_{0.05}Na_{0.95}Zr₂(PO₄)₃
Ag_{0.2}K_{0.8}Ti₂(PO₄)₃
Ag_{0.1} H_{0.9}Zr₂(PO₄)₃
Ag_{0.5}Na_{0.25}H_{0.25}Zr₂(PO₄)₃
Ag_{0.9}Na_{0.1}Zr₂(PO₄)₃
Ag_{0.7}Na_{0.3}Sn₂(PO₄)₃

A process for synthesizing a tetravalent metal phosphate-based antimicrobial agent represented by Formula (1) of the present invention employs a calcination method, a wet method, a hydrothermal method, etc., and it can be obtained easily by, for example, the process below.

Process for Synthesizing Tetravalent Metal Phosphate-Based Antimicrobial Agent

When it is synthesized by the calcination method, an alkali metal-containing compound such as lithium carbonate (Li₂CO₃) or sodium carbonate (Na₂CO₃) or an alkaline earth metal-containing compound, a tetravalent metal-containing compound such as zirconium oxide (ZrO₂), and a phosphoric acid group-containing compound such as ammonium dihydrogen phosphate (NH₄H₂PO₄) are mixed at a molar ratio of about 1:4:6, and the mixture is calcined at 1100° C. to 1400° C. to give a tetravalent metal phosphate-based compound represented by Formula (2) below.
Q′ₓM₂(PO₄)₃ (2)

Q′ in Formula (2) is at least one type of metal ion selected from the group consisting of an alkali metal ion, an alkaline earth metal ion, and ammonium ion, M is the same as that in Formula (1), and x is 1 when Q′ is monovalent and ½ when Q′ is divalent.

By immersing the compound represented by Formula (2) in an aqueous solution containing an appropriate concentration of silver ion at room temperature to 100° C., the compound represented by Formula (1) can be obtained.

When a compound represented by Formula (1) is synthesized by the wet method, phosphate ion and a tetravalent metal ion are reacted in water in the presence of at least one type of ion selected from the group consisting of an alkali metal ion, an alkaline earth metal ion, and ammonium ion to give a tetravalent metal phosphate, on which silver ion is supported.

More specifically, oxalic acid is added to an aqueous solution of zirconium oxynitrate and sodium nitrate while stirring, and phosphoric acid is then added. The pH of the reaction mixture is adjusted to about 3.5 using an aqueous solution of sodium hydroxide, and after it is heated and refluxed for 7 to 8 hours, a precipitate is filtered, washed with water, dried, and then ground to give a tetravalent metal phosphate (NaM₂(PO₄)₃) (M is the same as that in Formula (1), and is zirconium in this example. The compound may be a hydrate). By immersing this compound in an aqueous solution containing an appropriate concentration of an antimicrobial metal, an antimicrobial agent represented by Formula (1) is obtained. As described in Examples below, it is also possible to use zirconium oxychloride instead of zirconium oxynitrate.

The value of a in Formula (1) may be adjusted as appropriate according to required characteristics and application conditions. For example, by adjusting the concentration of silver ion in an aqueous solution in which the compound represented by Formula (2) is immersed, the duration and temperature of immersion in the aqueous solution, etc. it is possible to obtain compounds having different values for a of Formula (1).

In order for antimold, antibacterial, and antialgal properties to be exhibited in an antimicrobial resin composition or an antimicrobial fiber composition, which will be described below, it is desirable for a in Formula (1) to be large. If the value of a of Formula (1) is 0.001 or larger, sufficient antimold, antibacterial, and antialgal properties can be exhibited. However, if the value of a of Formula (1) is less than 0.01, it might become difficult for antimold, antibacterial, and antialgal properties to be exhibited for a long period of time, and it is therefore preferable for the value of a to be equal to or greater than 0.01. Furthermore, in order to maintain moldability and mechanical strength of an antimicrobial product to which the antimicrobial composition is added and in order for sufficient antimold, antibacterial, and antialgal properties to be exhibited for a long period of time, it is preferable for the value of a to be equal to or greater than 0.2. When cost effectiveness is taken into consideration, it is preferable for the value of a to be equal to or less than 0.7.

In order to improve the chemical stability and physical stability of the tetravalent metal phosphate-based antimicrobial agent represented by Formula (1) of the present invention so as to obtain an antimicrobial agent for which there is a high level of prevention of discoloration due to exposure to heat or light, it is preferable to subject this tetravalent metal phosphate-based compound to a calcination step after silver ion is supported.

It is possible by means of this calcination step to obtain an antimicrobial agent whose chemical stability and physical stability are outstandingly improved, and which does not discolor and has excellent weather resistance. In addition, since little of the moisture attached prior to calcination remains, the processability of a resin to which the antimicrobial agent is added is also improved. In this step, it is preferable to calcine the tetravalent metal phosphate-based compound having silver ion supported thereon at 500° C. to 1300° C., more preferably 600° C. to 1000° C., and particularly preferably 700° C. to 900° C. If calcination is carried out at a temperature of less than 500° C., there is a possibility that the effect of improving the chemical stability and physical stability of the antimicrobial agent might be insufficient, and if calcination is carried out at a temperature of higher than 1300° C., there is a possibility that the antimicrobial properties might deteriorate, or it might be difficult to obtain microparticles of the antimicrobial agent due to microparticles of the tetravalent metal phosphate-based compound fusing to each other. The calcination time is not particularly limited, and the effects of the present invention can normally be exhibited sufficiently by calcining for 1 to 20 hours. The rate of temperature increase and the rate of temperature decrease are not particularly limited either and are adjusted as appropriate while taking into consideration the capacity of a calcining furnace, the productivity, etc.

In order to obtain an antimicrobial agent having excellent antimicrobial properties and weather resistance, it is preferable to support hydrogen ion on the tetravalent metal phosphate-based antimicrobial agent of the present invention. When the tetravalent metal phosphate-based antimicrobial agent has ammonium ion, the ammonium ion is thermally decomposed to form hydrogen ion by carrying out the above-mentioned calcination step, and it is therefore possible to support hydrogen ion on the tetravalent metal phosphate-based antimicrobial agent by subjecting it to the calcination step. Preferred calcination conditions during this process are a temperature of 600° C. to 1100° C. and a time of about 0.5 to 2 hours.

On the other hand, when the tetravalent metal phosphate-based antimicrobial agent does not have ammonium ion or has only a very small amount thereof, it is preferable to add a step of supporting hydrogen ion on the tetravalent metal phosphate-based antimicrobial agent; as a typical method therefor there is, for example, a method in which a tetravalent metal phosphate-based compound is immersed in an acidic solution, and this method has high productivity compared with the above-mentioned method in which the tetravalent metal phosphate-based compound having ammonium ion is calcined. The tetravalent metal phosphate-based compound that is immersed in the acidic solution may or may not be one on which silver ion has been supported. Preferred specific examples of the acidic solution in which the tetravalent metal phosphate-based compound is immersed for supporting hydrogen ion include aqueous solutions of hydrochloric acid, sulfuric acid, and nitric acid. The acid concentration and the temperature of the acidic solution and the immersion time are not particularly limited but, in general, the higher the acid concentration and the higher the temperature, the shorter the time taken for supporting hydrogen ion, and the acid concentration is therefore preferably 0.1 N or higher, the treatment temperature is preferably 40° C. or higher, and more preferably at least 60° C. but not more than 100° C., and the immersion time is preferably at least 10 minutes, and more preferably at least 60 minutes.

The tetravalent metal phosphate-based antimicrobial agent used in the present invention is stable to exposure to heat or light, the structure and the composition thereof are completely unchanged even after heating at 500° C., and in some cases even at 800° C. to 1100° C., and the color is completely unchanged by irradiation with ultraviolet rays. Furthermore, the framework structure of the tetravalent metal phosphate-based antimicrobial agent used in the present invention is not changed even by contact with liquid water or in acidic solution. Therefore, there is no restriction on heating temperature or light-shielding conditions during the processing of various types of moldings or during the storage or, unlike conventional antimicrobial agents, use thereof.

The tetravalent metal phosphate-based antimicrobial agent is obtained by the above-mentioned synthetic process as polydisperse particles having a particle size distribution. The antimicrobial particles used in the present invention may be obtained by filtering these polydisperse antimicrobial particles so as to eliminate particles having a predetermined particle size or greater, thus removing coarse particles, which present a barrier when spinning fiber. The maximum particle size cut-off may be selected as appropriate and is, for example, 10 μm, 5 μm, or 2 μm.

The tetravalent metal phosphate-based antimicrobial particles used in the present invention have a maximum particle size of substantially equal to or less than 10 μm, preferably substantially equal to or less than 5 μm, and more preferably substantially equal to or less than 2 μm. ‘Substantially’ referred to here means that the proportion of the weight of the group of particles having a particle size that is equal to or less than the maximum particle size is at least 98% of the total weight of the particles, preferably at least 99%, and more preferably at least 99.5%. It is undesirable to use crude particles having a maximum particle size exceeding 10 μm since, for example, if they are added to a fiber, when melt spinning is carried out filter blockage and filament breakage easily occur. The average particle size is not particularly limited, but is preferably 0.1 to 5 μm, and more preferably in the range of 0.2 to 2 μm. If the microparticles have an average particle size of less than 0.1 μm, when mixed with a fiber resin they aggregate and thus easily form coarse particles, thereby causing filter blockage and filament breakage during melt spinning. Moreover, when the average particle size exceeds 5 μm, the same problem might occur. The average particle size referred to in the present invention means an average particle size on a volume basis measured by a laser diffraction method.

Inorganic Compound Particles

The inorganic compound particles used in the present invention are those having a Mohs hardness of equal to or less than 6. When the Mohs hardness is higher than 6, there is a possibility that wear of a guide might not be suppressed during spinning of a fiber. The inorganic compound particles used in the present invention preferably have a Mohs hardness of equal to or less than 6.0, and more preferably a Mohs hardness of 3.0 to 6.0.

With regard to the inorganic compound particles having a Mohs hardness of equal to or less than 6.0, colorless or white particles are preferable.

With regard to the inorganic compound particles having a Mohs hardness of equal to or less than 6.0, an alkaline earth metal salt of an inorganic acid, and a metal oxide are preferable, and those having a white color are particularly suitable. Specific examples thereof include calcium carbonate, magnesium carbonate, aluminum hydroxide, aluminum potassium sulfate, calcium sulfate, barium sulfate, MgO, calcium phosphates (Ca₃(PO₄)₂, CaHPO₄, etc.), talc, mica, anatase titanium dioxide, zinc oxide, colloidal silica, and aluminum silicate hydrate.

The tetravalent metal phosphate-based antimicrobial particles used in the present invention are not included in the inorganic compound particles having a Mohs hardness of equal to or less than 6.0.

Since the inorganic compound particles are exposed to a high temperature of 200° C. or greater during molding of a resin or spinning of a fiber, it is preferable for the inorganic compound particles not to have water of crystallization or a decomposable product in the compound, or if they have these, it is preferable for them not to release water or decompose until 300° C. and to have low hygroscopicity. Preferred examples thereof include calcium carbonate, calcium sulfate, barium sulfate, anatase titanium dioxide, and zinc oxide, and an anhydrous compound is preferably used. Furthermore, anatase titanium dioxide is more preferable since it is commonly used in fiber and has a high effect in preventing discoloration.

It is known that anatase titanium dioxide has photocatalytic activity. It is undesirable to use a photocatalytically active titanium dioxide in the antimicrobial composition of the present invention since, when it is added to a resin and molded or when a molding is used under an environment when there is exposure to light, there is a possibility of marked discoloration or degradation of the resin, and it is therefore preferable to use anatase titanium dioxide that has had its photocatalytic activity suppressed or eliminated by subjecting it to a surface treatment, etc.

In the same way as for the tetravalent metal phosphate-based antimicrobial particles, the inorganic compound particles are generally obtained as polydisperse particles having a particle size distribution. The inorganic compound particles used in the present invention may be obtained by filtering these polydisperse inorganic compound particles so as to eliminate particles having a predetermined particle size or greater, thus removing coarse particles, which present a barrier when spinning fiber. The maximum particle size cut-off may be selected as appropriate and is, for example, 10 μm, 5 μm, or 2 μm.

The inorganic compound particles used in the present invention have a maximum particle size of substantially equal to or less than 10 μm, preferably substantially equal to or less than 5 μm, and more preferably substantially equal to or less than 2 μm. The definition of ‘substantially’ is the same as that for the tetravalent metal phosphate-based antimicrobial particles. It is undesirable to use crude particles having a maximum particle size exceeding 10 μm since, for example, if they are added to a fiber, when melt spinning is carried out, filter blockage and filament breakage easily occur. The average particle size is not particularly limited, but is preferably 0.1 to 5 μm, and more preferably in the range of 0.2 to 2 μm. If the microparticles have an average particle size of less than 0.1 μm, when mixed with a fiber resin they aggregate and thus easily form coarse particles, thereby causing filter blockage and filament breakage during melt spinning. Moreover, when the average particle size exceeds 5 μm, the same problem might occur. The average particle size referred to here means 50 wt % diameter based on a particle size distribution curve.

More preferably, the average particle size is smaller than that of the tetravalent metal phosphate-based antimicrobial particles. If the average particle size is larger than that of the tetravalent metal phosphate-based antimicrobial particles represented by Formula (1), since it is difficult for the inorganic compound particles to stay attached to the surface of the tetravalent metal phosphate-based antimicrobial particles, there is a possibility that the processability of the antimicrobial resin composition obtained by adding the antimicrobial composition to a fiber or a film might not be improved.

In the present invention, the inorganic compound particles may be used singly or in a combination of two or more types.

Proportions of Antimicrobial Composition

With regard to preferred proportions of the tetravalent metal phosphate-based antimicrobial particles and the inorganic compound particles of the antimicrobial composition of the present invention, relative to 100 parts by mass (hereinafter simply called parts) of the total thereof, the inorganic compound particles make up 5 to 90 parts, preferably 30 to 80 parts, and more preferably 50 to 75 parts. If the proportion of the inorganic compound particles is less than 5 parts, the processability of the antimicrobial resin composition obtained by adding the antimicrobial composition to a fiber or a film might not be improved, and if the inorganic compound particles make up more than 90 parts, there is a possibility that it might be difficult for the tetravalent metal phosphate-based antimicrobial particles to exhibit an antimicrobial effect. In order to exhibit sufficient antimicrobial properties, the silver ion concentration in the antimicrobial composition is preferably at least 0.5 mass %, and more preferably at least 2 mass %.

Antimicrobial Resin Composition

The antimicrobial composition of the present invention may be added to a resin to give an antimicrobial resin composition.

Resin

The resins that can be used may be any of natural resins, semi-synthetic resins, and synthetic resins, and may be either thermoplastic resins or thermosetting resins. The resin may be any one of a molding resin, a fiber resin, and a rubber resin, and specific examples of the resin include molding resins such as polyethylene, polypropylene, vinyl chloride, ABS resin, nylon, polyester, polyvinylidene chloride, polyamide, polystyrene, polyacetal, polycarbonate, acrylic resin, fluorine resin, polyurethane elastomer, polyester elastomer, melamine resin, urea resin, ethylene tetrafluoride resin, unsaturated polyester resin, epoxy resin, urethane resin, and phenolic resin; fiber resins such as nylon, polyethylene, rayon, acetate, acrylic, polyvinyl alcohol, polypropylene, cupra, triacetate, and vinylidene; and rubber resins such as natural rubber, silicone rubber, SBR (styrene butadiene rubber), CR (chloroprene rubber), EPM (ethylene propylene rubber), FPM (fluorine rubber), NBR (nitrile rubber), CSM (chlorosulfonated polyethylene rubber), BR (butadiene rubber), IR (synthetic natural rubber), IIR (butyl rubber), urethane rubber, and acrylic rubber.

Proportions of Antimicrobial Resin Composition

A preferred proportion of the antimicrobial composition in the antimicrobial resin composition is 0.01 to 10 parts by mass relative to 100 parts by mass of the antimicrobial resin composition. If it is less than 0.01 parts by mass, there is a possibility that it might be difficult for the antimicrobial resin composition to exhibit sufficient antimicrobial properties, and if it is added in a proportion exceeding 10 parts by mass, there is no greater improvement in the antimicrobial properties, and instead there is a possibility that other physical properties of the antimicrobial resin composition might be degraded. In order for sufficient antimicrobial properties to be exhibited, the silver ion concentration in the antimicrobial resin composition is preferably at least 0.001 mass %. When the composition is used as a master batch, the proportion of the antimicrobial composition is preferably 10 to 35 parts by mass, and more preferably 10 to 25 parts by mass.

The antimicrobial resin composition can easily be made into an antimicrobial product by mixing the antimicrobial composition and the resin at an appropriate temperature or pressure according to the characteristics of the resin. Specific procedures therefor may be carried out by standard methods, and various types of form such as lump, film, filament, pipe, or a composite thereof, can be molded.

The form in which the antimicrobial composition of the present invention is used is not particularly limited, and it may be mixed with another component as appropriate according to the intended application or used as a composite with another material. Examples of the various types of form include a powder, a powder-containing dispersion, powder-containing particles, a powder-containing paint, a powder-containing fiber, a powder-containing paper, a powder-containing plastic, a powder-containing film, and a powder-containing aerosol, and the composition may be used as necessary in combination with various types of additives or materials such as an organic antimold agent, a deodorant, a flame retardant, an antistatic agent, a pigment, an inorganic ion exchanger, a corrosion inhibitor, a UV absorber, an antioxidant, a negative ion ceramic, and a construction material.

The antimicrobial resin composition comprising the antimicrobial composition of the present invention can be utilized as a starting material for an antimicrobial product in various types of fields where antimold, antialgal, and antibacterial properties are required. Specific representative examples of applications include, as resin moldings, moldings used in electrical appliances such as refrigerators, microwave ovens, or televisions, medical equipment, brushes, food containers, chopping boards, draining baskets; various types of wrapping materials such as food wrapping film; and film for medical use. In addition, it may be used as a paint such as a preservative paint or an antimold paint. Examples of fiber products include bed linen, towels, napkins, masks, stockings, tights, socks, work clothing, medical clothing, medical linen, sports clothing, bandages, fishing nets, curtains, carpets, underwear, and air filters. Examples of rubber products include various types of tubes, gaskets, and belts.

The antimicrobial resin composition thus obtained has good processability and can therefore be used in various types of applications and, in particular, there is a large effect in improving the wear of an equipment part such as a guide in a spinning process, and higher order processability such as spinning, twining, knitting, or weaving during a process to form a final product can be improved. In this case, it is preferable to use the composition for a filament having a narrow single filament fineness, and it is more preferable to use it for a filament of 5 d or less, and particularly 4 d or less since a large effect can be exhibited. In order to exhibit the effects of the present invention, the fiber structure is preferably a filament, and more preferably a multifilament since a thin filament can be used.

The resin used for the antimicrobial resin composition for a fiber is not particularly limited, but is preferably a polyamide resin, a polyester resin, a polyurethane resin, a polypropylene resin, etc.

Spinning for the antimicrobial resin composition of the present invention may be carried out by a standard method, and the method may be selected according to the application and purpose. It is particularly suitable when the discharge rate per mouthpiece is large or when the windup speed is high. In particular, a very large effect can be exhibited when the spinning speed is 3500 m/minute or higher.

The antimicrobial compositions of the present invention are useful as antimicrobial agents applied to materials such as various types of rubber and plastic, moldings such as films and sheets formed therefrom, and various types of fiber, paper, leather, paint, adhesives, heat-insulating materials, caulking materials, etc. In particular, an antimicrobial composition can be provided in fiber applications that does not cause filter pressure increase or filament breakage during melt spinning, can suppress the wear of a guide more than a case in which the tetravalent metal phosphate-based antimicrobial particles are used alone, has excellent processability during fiber spinning, and can impart excellent antimicrobial properties. An antimicrobial fiber can easily be obtained from this composition.

### EXAMPLES

The present invention is explained in further detail below by reference to examples, but the present invention should not be construed as being limited by the examples below and can be modified appropriately in ways that conform to the scope of the preceding and following descriptions, and these modifications are also included within the technical scope of the present invention. ‘%’ means ‘mass %’.

### Synthetic Example 1

### Synthesis of Tetravalent Metal Phosphate

Oxalic acid (0.1 mol) was added to an aqueous solution of zirconium oxychloride (0.2 mol) while stirring, and phosphoric acid (0.3 mol) was then added thereto (the equivalent weight of zirconium ion per equivalent weight of phosphate ion is 0.67). The pH of the solution was adjusted to about 3.5 with an aqueous solution of sodium hydroxide, and after the solution was heated and refluxed at 95° C. for 20 hours, a precipitate that formed was filtered, washed with water, dried, and then ground to give a network-form zirconium sodium phosphate (NaZr₂(PO₄)₃ 1.1H₂O) (Na type zirconium phosphate, Mohs hardness about 8, average particle size 0.9 μm).

### Synthetic Example 2

### Synthesis of Tetravalent Metal Phosphate-Based Antimicrobial Particles-1

The Na type zirconium phosphate powder obtained in Synthetic Example 1 was added to a 1N nitric acid solution containing silver ion and stirred at 60° C. for 10 hours. The slurry thus obtained was then filtered and washed well with pure water, further dried while heating at 110° C. overnight, and then calcined at 750° C. for 4 hours to give antimicrobial tetravalent metal phosphate-based antimicrobial particles (X) (silver content: 3.7 mass %).

### Synthetic Example 3

### Synthesis of Tetravalent Metal Phosphate-Based Antimicrobial Particles-2

The procedure of Synthetic Example 2 was repeated using the Na type zirconium phosphate powder obtained in Synthetic Example 1 except that the concentrations of silver and nitric acid were changed, thus giving tetravalent metal phosphate-based antimicrobial particles (Y) (silver content: 10 mass %).

Values for the physical properties of the two types of tetravalent metal phosphate-based antimicrobial particles (X) and (Y) obtained in Synthetic Example 2 and Synthetic Example 3 are given in Table 1.

TABLE 1

| | Average | Maximum | | |
|---|---|---|---|---|
| | particle | particle | Mohs | Silver |
| | size | size | hardness | content % |
| X | 0.9 μm | 1.3 μm | About 8 | 3.7% |
| Y | 0.9 μm | 1.3 μm | About 8 | 10.0% |

### Example 1

The antimicrobial phosphate compound (Y) obtained in Synthetic Example 3 and titanium oxide TA-300 (Mohs hardness 5.5, average particle size 0.4 μm), which is anatase titanium oxide, manufactured by Fuji Titanium Industry Co., Ltd. were mixed at a ratio of 3:7 to give an antimicrobial composition (A).

### Example 2

The antimicrobial phosphate compound (Y) obtained in Synthetic Example 3 and calcium carbonate Caltex 5 (Mohs hardness 3, average particle size 0.9 μm) manufactured by Maruo Calcium Co., Ltd. were mixed at a ratio of 3:7 to give an antimicrobial composition (B).

### Comparative Example 1

The antimicrobial phosphate compound (Y) obtained in Synthetic Example 3 and calcium carbonate Super SS (Mohs hardness 3, average particle size 2.2 μm, maximum particle size 15 μm) manufactured by Maruo Calcium Co., Ltd. were mixed at a ratio of 3:7 to give an antimicrobial composition (a).

### Comparative Example 2

The antimicrobial phosphate compound (Y) obtained in Synthetic Example 3 and titanium oxide CR-60-2 (Mohs hardness 6.5, average particle size 0.2 μm), which is rutile titanium oxide, manufactured by Ishihara Sangyo Kaisha Ltd. were mixed at a ratio of 3:7 to give an antimicrobial composition (b).

Values for the physical properties of antimicrobial compositions A, B, a, and b obtained in Examples 1 and 2 and Comparative Examples 1 and 2 are given in Table 2.

The particle size of the antimicrobial composition prepared in Examples 1 and 2 and Comparative Examples 1 and 2 was measured by a laser diffraction particle size distribution analyzer.

TABLE 2

| | Anti- | | | | |
|---|---|---|---|---|---|
| | microbial | | Average | Maximum | |
| | phosphate | Inorganic | particle size | particle size | |
| | compound | compound | of | of | Silver |
| | particles | particles | composition | composition | content |
| A | Y 30% | Anatase titanium | 0.5 μm | 1.3 μm | 3.0% |
| | | oxide 70% | | | |
| B | Y 30% | Calcium | 0.9 μm | 2.5 μm | 3.0% |
| | | carbonate 70% | | | |
| a | Y 30% | Calcium | 1.5 μm | 15 μm | 3.0% |
| | | carbonate 70% | | | |
| b | Y 30% | Rutile titanium | 0.5 μm | 1.3 μm | 3.0% |
| | | oxide 70% | | | |

### Example 3

Spinning Test of Antimicrobial Resin Composition

The antimicrobial composition A was added at 10% to a polyester resin (MA2103, manufactured by Unitika Ltd.) to give a master batch (antimicrobial resin composition). This was then mixed with polyester resin pellets to give an antimicrobial resin composition having an antimicrobial composition content of 1.0 mass %. The antimicrobial resin composition was subjected to melt spinning using a multi filament spinner at a spinning temperature of 275° C. and a windup speed of 4000 m/minute, and a 24 filament antimicrobial polyester fiber having 2 denier/filament was wound up in drum form to give an antimicrobial polyester fiber. Filament formation properties were evaluated with respect to filter pressure increase, the number of filament breakages, and the degree of the wear of a ceramic guide made of alumina during this process (each sample: 10 kg winding×4). The results are given in Table 3.

The antimicrobial polyester fiber thus obtained was refined, and antimicrobial properties were evaluated. The results are given in Table 3.

The antimicrobial properties were evaluated in accordance with a quantitative test of JIS L 1902-1998, and the test was carried out using Staphylococcus aureus. When the antimicrobial activity was equal to or greater than 2.2, it was evaluated as having antimicrobial properties.

### Example 4

Spinning Test of Antimicrobial Resin Composition

The procedure of Example 3 was repeated except that the antimicrobial composition B was used instead of the antimicrobial composition A, and the spinning properties were evaluated. The results are given in Table 3.

### Comparative Example 3

Spinning Test of Antimicrobial Resin Composition

The procedure of Example 3 was repeated except that the tetravalent metal phosphate-based antimicrobial particles (X) prepared in Synthetic Example 2 were used instead of the antimicrobial composition A, and the spinning properties were evaluated. The results are given in Table 3.

### Comparative Example 4

Spinning Test of Antimicrobial Resin Composition

The procedure of Example 3 was repeated except that the antimicrobial composition a prepared in Comparative Example 1 was used instead of the antimicrobial composition A, and the spinning properties were evaluated. The results are given in Table 3.

### Comparative Example 5

Spinning Test of Antimicrobial Resin Composition

The procedure of Example 3 was repeated except that the antimicrobial composition b prepared in Comparative Example 2 was used instead of the antimicrobial composition A, and the spinning properties were evaluated. The results are given in Table 3.

TABLE 3

| | Antimicro- | | Filter | | Degree | Anti- |
|---|---|---|---|---|---|---|
| | bial agent | | pressure | Number of | of wear | micro- |
| | composition, | Mass | increase | filament | of | bial |
| | etc. | % | (kg/cm²) | breakages | guide | activity |
| Ex. 3 | A | 1.0 | 0.2 | None | Little | >5.2 |
| Ex. 4 | B | 1.0 | 0.2 | None | Little | >5.2 |
| Comp. | X | 1.0 | 0.3 | None | Large | >5.2 |
| Ex. 3 | | | | | | |
| Comp. | a | 1.0 | 2.0 | 10 | Little | >5.2 |
| Ex. 4 | | | | | | |
| Comp. | b | 1.0 | 1.5 | None | Med- | >5.2 |
| Ex. 5 | | | | | ium | |

As is clear from the results in Table 3, it was found that the antimicrobial polyester fibers of Examples that satisfy the requirements of the present invention do not cause an increase in filter pressure or filament breakage during spinning, guide wear can be suppressed more than in a case in which the tetravalent metal phosphate-based antimicrobial particles are used alone, and the processability during fiber spinning is excellent. It was also found that excellent antimicrobial properties are exhibited. In contrast to the above, when the requirements of the present invention are not satisfied, it was found that the fiber spinning processability is poor.

### Example 5

Molding Test of Vinylidene Chloride Film

The antimicrobial composition A was added to a vinylidene chloride resin (Krehalon, manufactured by Kureha Chemical Industry Co., Ltd.) in amounts of 50 and 100 mg/m², and antimicrobial films having a film thickness of 4, 10, 18, and 44 μm were prepared by a T die method (operating conditions: extrusion temperature 200° C.). These antimicrobial films were examined with respect to the presence or absence of aggregates, film tearing, and antimicrobial activity. The results for an antimicrobial film having a content of the antimicrobial composition A of 100 mg/m² and a film thickness of 4 μm are given in Table 4.

The antimicrobial properties were evaluated in accordance with JIS Z 2801, and a test was carried out using Staphylococcus aureus. When the antimicrobial activity was equal to or greater than 2.0, it was evaluated as having antimicrobial properties. The results are given in Table 4.

### Example 6

Molding Test of Vinylidene Chloride Film

The procedure of Example 5 was repeated except that the antimicrobial composition B prepared in Example 2 was used instead of the antimicrobial composition A, and film moldability and antimicrobial activity were evaluated. The results for an antimicrobial film having a content of the antimicrobial composition B of 100 mg/m² and a film thickness of 4 μm are given in Table 4.

### Comparative Example 6

Molding Test of Vinylidene Chloride Film

The procedure of Example 5 was repeated except that the antimicrobial composition a prepared in Comparative Example 1 was used instead of the antimicrobial composition A, and film moldability and antimicrobial activity were evaluated. The results for an antimicrobial film having a content of the antimicrobial composition a of 100 mg/m² and a film thickness of 4 μm are given in Table 4.

TABLE 4

| | | Presence or | | |
|---|---|---|---|---|
| | Antimicrobial | absence of | Film | Antimicrobial |
| | composition | aggregates | tearing | activity |
| Ex. 5 | A | None | None | >5.2 |
| Ex. 6 | B | None | None | >5.2 |
| Comp. | a | Some | Some | >5.2 |
| Ex. 6 | | | | |

As is clear from the results in Table 4, it was found that the antimicrobial vinylidene chloride films of Examples that satisfy the requirements of the present invention do not cause aggregation or film tearing, and have excellent processability during molding. It was also found that excellent antimicrobial properties are exhibited.

The results for films having different antimicrobial composition contents and films having different film thicknesses were the same as those in Table 4 above.

1. An antimicrobial composition comprising:
tetravalent metal phosphate-based antimicrobial particles represented by Formula (1); and
inorganic compound particles having a Mohs hardness of equal to or less than 6;
the maximum particle size of these particles being substantially equal to or less than 10 μm
AgₐQ_{b}M₂(PO₄)₃.nH₂O (1)
(in the formula, Q denotes at least one type of ion selected from the group consisting of an alkali metal ion, an alkaline earth metal ion, ammonium ion, and hydrogen ion, M is a tetravalent metal ion, n is a number satisfying 0≦n≦6, a and b are both positive numbers, m is the valence of Q, and a+mb=1).

2. The antimicrobial composition according to claim 1, wherein the tetravalent metal phosphate-based antimicrobial particles and the inorganic compound particles have an average particle size of 0.1 to 5 μm.

3. The antimicrobial composition according to claim 1, wherein the average particle size of the inorganic compound particles is smaller than the average particle size of the tetravalent metal phosphate-based antimicrobial particles.

4. The antimicrobial composition according to claim 1, wherein the inorganic compound particles are anatase titanium dioxide having no photocatalytic activity.

5. The antimicrobial composition according to claim 1, wherein the mixing ratio of the tetravalent metal phosphate-based antimicrobial particles and the inorganic compound particles is 95:5 to 10:90.

6. An antimicrobial product comprising the antimicrobial composition according to claim 1.

7. An antimicrobial product comprising the antimicrobial composition according to claim 2.

8. An antimicrobial product comprising the antimicrobial composition according to claim 3.

9. An antimicrobial product comprising the antimicrobial composition according to claim 4.

10. An antimicrobial product comprising the antimicrobial composition according to claim 5.

11. The antimicrobial product according to claim 6, wherein the antimicrobial product is an antimicrobial fiber or an antimicrobial film.

12. The antimicrobial product according to claim 7, wherein the antimicrobial product is an antimicrobial fiber or an antimicrobial film.

13. The antimicrobial product according to claim 8, wherein the antimicrobial product is an antimicrobial fiber or an antimicrobial film.

14. The antimicrobial product according to claim 9, wherein the antimicrobial product is an antimicrobial fiber or an antimicrobial film.

15. The antimicrobial product according to claim 10, wherein the antimicrobial product is an antimicrobial fiber or an antimicrobial film.
